(19) United States
(12) Patent Application Publication
(10) Pub. No.: US 2016/0120928 A1
(43) Pub. Date: May 5, 2016

(54) NASAL SPRAY

(71) Applicant: ZENT, INC., Palo Alto, CA (US)

(72) Inventors: Wiwik S. Watanabe, Union City, CA (US); Thomas A. Armer, Los Altos, CA (US); Jian Zhang, Union City, CA (US)

(73) Assignee: ZENT, INC., Palo Alto, CA (US)

(21) Appl. No.: 14/929,319

(22) Filed: Oct. 31, 2015

Related U.S. Application Data
(60) Provisional application No. 62/074,143, filed on Nov. 3, 2014.

(51) Int. Cl. A61K 36/752; A61K 36/886; A61K 9/00

(52) U.S. Cl. CPC A61K 36/752; A61K 9/0043; A61K 36/886

(57) ABSTRACT

The invention relates to novel formulations for treating rhinitis, sinusitis, and allergies with a fast onset of action, long duration (about once or twice daily), with no or minimal irritation, and without rebound congestion.

### FIELD OF THE INVENTION

The invention relates to novel formulations for treating rhinitis, sinusitis, and allergies with a fast onset of action, long duration (about once or twice daily), with no or minimal irritation, and without rebound congestion. The formulations contain natural ingredients with anti-inflammatory, anti-histamine, analgesic, antiseptic, anti-oxidant, or soothing effects for nasal administration.

### BACKGROUND OF THE INVENTION

Rhinitis is a common symptom of upper respiratory tract infections, allergies, hay fever, and irritations from inhaled particulates or pollutants. It is usually caused by nasal mucosal inflammation and secretion. The underlining diseases include various types of rhinitis and sinusitis (also called rhinosinusitis), which could be either acute or chronic. Surveys revealed that 60% of allergic rhinitis patients and 70% of rhinosinusitis patients suffer from rhinitis. Stewart et al., Int. J. Gen. Med., 3:37-45 (2010).

Rhinitis was responsible for millions of physicians' office visits in 2013. Rhinitis patients may suffer significant economic and quality-of-life burdens. For instance, sinusitis affects 37 million people each year, making it one of the most common health problems in the U.S. It is more prevalent than heart disease and asthma and has a greater impact on quality of life than chronic back pain or congestive heart failure. Symptoms may significantly affect people physically, functionally, and emotionally. See Medical Center of McKinney website, http://medicalcenterofmckinney.com/service/sinus-center. Sinusitis affects approximately 14% of the adult U.S. population. Chronic sinusitis (not including acute sinusitis) results annually in an estimated 18-22 million physician office visits. Direct healthcare expenditures due to sinusitis cost are well over $8 billion each year. Id.

Sinusitis causes the sinuses to become inflamed. The sinuses are air spaces behind the bones of the upper face, between the eyes and behind the forehead, nose and cheeks. Normally, the sinuses drain through small openings into the inside of the nose. Anything that blocks the flow may cause a buildup of mucus in the sinuses. The blockage and inflammation of the sinus membranes can be infectious or non-infectious. The symptoms caused by sinusitis may be quite uncomfortable and may include: facial pain, pressure, congestion or fullness, difficulty breathing through the nose, discharge of yellow or green mucus from the nose, tooth pain, loss of the sense of smell or taste, headache, fatigue, sore throat, and bad breath.

Standard methods of treatments include nasal steroids and nasal decongestant. Topical corticosteroids administered as nasal spray or dry powder are commercially available for prophylactic use. However, they have slow onset of action (12 hours) and require days or even weeks to reach their maximum efficacy. Typically, nasal decongestants relieve rhinitis by causing vasoconstriction. This class of drugs is known to relieve rhinitis relatively quickly (within half an hour) and last for hours. However prolonged use (>10 days) of topical vasoconstrictors may lead to rebound swelling of the nasal mucosa and rhinitis medicamentosa (drug-induced rhinitis). van Cauwenberge, et al., Allergy 55:116-134 (2000).

### SUMMARY OF THE INVENTION

The invention relates to novel formulations for treating rhinitis, sinusitis, and allergies with a fast onset of action, and long duration (once daily, twice daily, more than twice daily, or on an “as needed” basis) without irritation and rebound congestion.

Thus, the invention provides a composition for nasal administration, comprising about 0.10% to 10% volume to volume (v/v) lemon oil, about 1.0% to 10% v/v emulsifier, about 1.0% to 10% v/v alcohol, and about 0.10 to 2.5% v/v of a citric acid-containing solution, wherein the composition reduces the severity of rhinitis. In a preferred embodiment, the composition further comprises between about 0.5× and 10× Aloe Vera solution. In a more preferred embodiment, the Aloe Vera solution is at about a 1× or a 5× concentration.

In another preferred embodiment, the citric acid containing solution contains flavonoids. In a more preferred embodiment, the citric acid containing solution is lemon juice. In another more preferred embodiment, the citric acid containing solution is lemon juice at a concentration of about 2.0% v/v. In another more preferred embodiment, the lemon oil is at a concentration of about 0.24% v/v. In another more preferred embodiment, the lemon oil is at a concentration of about 0.48% v/v.

In another preferred embodiment, kushen, saphora flavoscens extract (containing matrine and kushenin) is included at a concentration 0.001% to 0.5% w/v.

In a most preferred embodiment, the emulsifier is polysorbate 80 at a concentration of about 3.0%, the alcohol is ethanol at a concentration of about 3.0%, the citric acid-containing solution is lemon juice at a concentration of about 2.0% v/v, and further comprising Aloe Vera at a concentration of about 5×.

The invention provides a method for treating rhinitis, sinusitis and allergies in a subject, comprising administering the compositions disclosed herein to the subject. The invention also provides for the use of the compositions disclosed herein for the manufacture of a medicament for treating rhinitis, sinusitis and allergies in a subject.

### BRIEF DESCRIPTION OF THE DRAWINGS

FIG. 1 shows a table describing the contents of five formulations. The formulations were rated by one or two subjects for the speed of onset of action, duration of nasal relief, the degree of irritation, and an efficacy rating on a 1-5 scale.

FIG. 2 shows a table describing seven formulations and their evaluation by the two subjects for irritation, taste, and smell.

FIG. 3 shows a table describing four formulations optimized for efficacy, smell, and irritation. They were evaluated by the two subjects.

### DETAILED DESCRIPTION OF THE INVENTION

The invention relates to novel formulations for treating rhinitis, sinusitis, or allergies with a fast onset of action. The invention may be dosed once, twice or more than twice daily with reduced or ablated irritation or rebound congestion. It has been surprisingly found that certain formulations of lemon juice (a citric acid-containing solution), lemon essential oil, polysorbate 80, ethanol, Aloe Vera, and water provide long-lasting relief from rhinitis without rebound swelling or resistance to treatment. Additionally, the formulations do not hurt and have a pleasant taste, smell and feel. While many of the ingredients disclosed herein are known in the art, the invention teaches novel combinations of the ingredients in particular concentrations that provide unexpectedly effective relief to subjects suffering from rhinitis, sinusitis, and allergies that is greater than that provided by any of the individual ingredients or by the formulations disclosed in the prior art.

While not wishing to be bound by theory, each of the ingredients in the novel combinations provides different benefits. Lemon juice neutralizes histamine to provide immediate relief Lemon essential oil and kushen extract are long-acting ingredients that act on multiple allergy pathways to prevent re-occurrence of allergy symptoms. Emulsifiers (surfactants) encapsulate essential oil into an oil-in-water emulsion and thus provides an even distribution of essential oil in the nasal cavity. Alcohol is a co-surfactant for the emulsion. Aloe Vera provides soothing effects that reduce the irritation caused by the low pH resulting from the lemon juice. Water is used as a solvent.

### DEFINITIONS

The term “absorption” is the movement of a therapeutic formulation into the bloodstream or a targeted tissue. A drug needs to be introduced via some route of administration (e.g. nasal, oral, topical or dermal) or in a specific dosage form such as a tablet, capsule or liquid. The formulations of the invention may act both locally in the nasal cavity and more systemically by reducing histamines or allergic responses.

An “effective amount” refers to an amount of a therapeutic formulation that is effective at dosages and for periods of time necessary to achieve the desired therapeutic or prophylactic result. A “therapeutically effective amount” of a therapeutic formulation may vary according to factors such as the disease state, age, sex, and weight of the individual, and its ability to elicit a desired response in the individual. A therapeutically effective amount may be measured, for example, by more rapid recovery, or amelioration, improvement or elimination of symptoms, or other acceptable biomarkers or surrogate markers. A therapeutically effective amount is also one in which any toxic or detrimental effects of the therapeutic compound are outweighed by the therapeutically beneficial effects. A “prophylactically effective amount” refers to an amount of a therapeutic formulation that is effective, at dosages and for periods of time necessary, to achieve the desired prophylactic result. Typically, but not necessarily, since a prophylactic dose is used in subjects prior to or at an earlier stage of disease, the prophylactically effective amount will be less than the therapeutically effective amount.

“Allergies” are hypersensitivity disorders of the immune system. Symptoms may include red eyes, itchiness, runny nose, eczema, hives, or asthma. Allergic reactions occur when a person's immune system reacts to normally harmless substances in the environment. A substance that causes a reaction is called an allergen.

“Flavonoids” are naturally-occurring class of compounds that are often found in citrus fruits and other natural sources. They include, for example, flavonols, flavones, anthocyanidins, isoflavones, flavanones, catechins, methoxylated flavones, polymethoxylated flavones, derivatives thereof, and metabolites thereof. Tripoli, et al., Food Chemistry, 104:466-479 (2007). The foregoing are incorporated by reference herein in their entirety. Citrus flavonoids may have anti-inflammatory activities such as inhibiting enzymes (protein kinase C, phosphodiesterase, phospholipase, lipoxygenase, and cyclooxygenase). They may also affect B and T cell activation.

“Homologs” are bioactive molecules that are similar to a reference molecule at the chemical, functional, or structural level. Homologs may be chemically-modified derivatives or metabolites.

An “individual,” “subject” or “patient” is a vertebrate. In certain embodiments, the vertebrate is a mammal. Mammals include, but are not limited to, primates (including human and non-human primates) and rodents (e.g., mice, hamsters, guinea pigs, and rats). In certain embodiments, a mammal is a human. A “control subject” refers to a healthy subject who has not been diagnosed as having a disease, dysfunction, or condition that has been identified in an individual, subject, or patient. A control subject does not suffer from any sign or symptom associated with the disease, dysfunction, or condition.

A “medicament” is an active therapeutic formulation that has been manufactured for the treatment of a disease, disorder, or condition.

“Patient response” or “response” can be assessed using any endpoint indicating a benefit to the patient, including, without limitation, (1) inhibition, to some extent, of rhinitis, including slowing down and complete arrest; (2) reduction in the number of disease episodes and/or symptoms; (3) decrease of an autoimmune condition; (4) favorable change in the expression of a biomarker associated with the nasal condition; or (5) relief, to some extent, of one or more symptoms associated with the condition; (6) increase in the length of nasal relief following treatment.

As used herein, a “pharmaceutically acceptable carrier” or “therapeutically effective carrier” is aqueous or nonaqueous (solid), for example alcoholic or oleaginous, or a mixture thereof, and can contain a surfactant, emollient, lubricant, stabilizer, dye, perfume, preservative, acid or base for adjustment of pH, a solvent, emulsifier, gelling agent, moisturizer, stabilizer, wetting agent, time release agent, humectant, or other component commonly included in a particular form of pharmaceutical composition. Pharmaceutically acceptable carriers are well known in the art and include, for example, aqueous solutions such as water or physiologically buffered saline or other solvents or vehicles such as glycols, glycerol, and oils such as olive oil or essential oils. A pharmaceutically acceptable carrier can contain physiologically acceptable compounds that act, for example, to stabilize or to increase the absorption of specific formulation, for example, carbohydrates, such as glucose, sucrose or dextrans, antioxidants such as ascorbic acid or glutathione, chelating agents, low molecular weight proteins or other stabilizers or excipients.

Rhinitis is irritation and inflammation of the mucous membrane inside the nose. Common symptoms of rhinitis are congestion, a stuffy nose, runny nose, and post-nasal drip. A common type, allergic rhinitis, may be triggered by airborne allergens such as pollen and dander. Allergic rhinitis may cause additional symptoms such as sneezing, nasal itching, coughing, headache, fatigue, malaise, and cognitive impairment.

As used herein, “treatment” refers to clinical or home intervention that alters the natural course of rhinitis and can be performed before or during the course of these conditions. Desirable effects of treatment include preventing the occurrence or recurrence of these conditions or symptoms thereof, alleviating the condition or symptom of the condition, diminishing any direct or indirect pathological consequences of the condition, decreasing the rate of progression, and ameliorating the condition. In some embodiments, methods and compositions of the invention are useful in attempts to delay development of the condition.

The formulations of the invention may comprise many citrus flavonoids, including those of the aglycone and glycoside subgroups. Examples of aglycone citrus flavonoids include naringenin, hesperetin, isosakranetin, heridictyol, apigenin, luteolin, diosmetin, Quercetin, and kämpferol. Examples of glycoside citrus flavonoids include naringin, neohesperidin, poncirin, neocriocitrin, narirutin, hesperidin, didymin, eriocitrin, and diosmin. They may be chemically synthesized or extracted from natural sources such as lemon, bitter orange, and grapefruit or bergamot seeds.

The formulation of the invention may comprise isoflavoniods and alkaloids derived from flavascens including kushenin and matrine.

The formulations of the invention might comprise additional active ingredients or flavor enhancers. Examples include compounds, extracts or essential oils from one or more of the following sources: ascorbic acid, vitamin E, omega 3, salt water, menthol, agar essential oil, agar extract, ajwain, aloe vera, amyris, angelica root, anise, balsam, basil, bay rum, bergamot, black pepper, buchu, butterbur, cajeput, cannabis flower, caraway, cardamom seed, carrot seed, cedarwood, Cedarleaf, chamomile, cinnamon, cistus, citrus vulgaris, citronella, clary sage, clove leaf, coriander, costmary, cranberry seed, cumin/black seed, cypress, davana, dill, eucalyptus, fennel seed, fenugreek, frankincense, galbanum, geranium, ginger, grapefruit, grape seed (e.g. Vitis vinifera), henna, jasmine, juniper berry, lavender, lemon, lemongrass, lime, litsea cubeba, lobelia/neem, melissa (Lemon balm), mentha arvensis/Mint, mugwort, mustard, myrrh, neroli, nutmeg, orange, oregano, orris, parsley, patchouli, perilla, pennyroyal, peppermint, pine, rose, rosehip, rosemary, rosewood, sage, sandalwood, sassafras, savory, schisandra, spearmint, star anise, tarragon, tea tree, thyme, vetiver, yarrow ylang-ylang, and other herbs, natural flavorings, or artificial flavorings known in the art.

The concentration of lemon oil in the formulations of the invention may vary so long as it is sufficient to provide long-acting relief for nasal inflammation and allergies. Thus, the concentration of lemon oil may be about 0.10% to 10% v/v lemon oil. In preferred embodiments, the concentration may be about 0.10%, 0.20%, 0.30% 0.40%, 0.5%, 0.60%, 0.70%, 0.80%, 0.90%, or 1.0% v/v. In particularly preferred embodiments, the lemon oil concentration is 0.24% or 0.48% v/v. In other preferred embodiments, the concentration may be between about 1.0% to 2.0%, 2.0% to 3.0%, 3.0% to 4.0%, 4.0% to 5.0%, 5.0% to 6.0%, 6.0% to 7.0%, 7.0% to 8.0%, 8.0% to 9.0%, or 9.0% to 10.0% v/v, or increments therebetween.

The formulations of the invention comprise an emulsifier or surfactant. In some embodiments, the concentration of the emulsifier is about 1.0% to 10% v/v. In preferred embodiments, the concentration may be about 1.0% to 2.0%, 2.0% to 3.0%, 3.0% to 4.0%, 4.0% to 5.0%, 5.0% to 6.0%, 6.0% to 7.0%, 7.0% to 8.0%, 8.0% to 9.0%, or 9.0% to 10.0% v/v, or increments therebetween. In a more preferred embodiment, the concentration is about 3.0%.

Emulsifiers for pharmaceutical, nutraceutical, and other human consumption are well-known in the art. Exemplary emulsifiers for use in the formulations of the invention include food emulsifiers such as egg yolk (lecitihin), mustard (mucilage), soy lecithin, pickering stabilization, sodium stearoyl lactylate, and DATEM (Diacetyl Tartaric Acid Ester of Monoglyceride) and anise. Other emulsifiers include emulsifying wax, cetearyl alcohol, polysorbate 20, and ceteareth 20.

The formulations of the invention comprise alcohol as surfactant or penetration enhancer. In some embodiments, the concentration of alcohol is about 1.0% to 10% v/v. In preferred embodiments, the concentration of alcohol may be about 1.0% to 2.0%, 2.0% to 3.0%, 3.0% to 4.0%, 4.0% to 5.0%, 5.0% to 6.0%, 6.0% to 7.0%, 7.0% to 8.0%, 8.0% to 9.0%, or 9.0% to 10.0% v/v, or increments therebetween. In a more preferred embodiment, the concentration is about 3.0%. Exemplary alcohols include ethanol, isopropyl alcohol, and benzyl alcohol.

The formulations of the invention comprise kushen extract at a final concentration of about 0.001% to 0.5% w/v. In a preferred embodiment, the kushen extract is at a final concentration of about 0.001% to 0.4% w/v. In another preferred embodiment, the kushen extract is at a final concentration of about 0.001% to 0.3% w/v. In another preferred embodiment, the kushen extract is at a final concentration of about 0.001% to 0.2% w/v. In another preferred embodiment, the kushen extract is at a final concentration of about 0.001% to 0.1% w/v. In another preferred embodiment, the kushen extract is at a final concentration of about 0.001% to less than 0.1% w/v.

The examples provided herein recite the use of 10% citric acid or filtered lemon juice as the citric acid containing component of the formulations. Any synthetic or natural citric acid solution may be used, however, so long as it is suitable for human consumption and provides the therapeutic benefits described herein. Natural sources of citric acid that may be suitable for the formulations of the invention include lemons, limes, oranges, strawberries, raspberries, gooseberries, cranberries, pineapples, cherries, tomatoes, some varieties of peppers, artichokes, and some varieties of lettuce. The concentration of citric acid containing solutions in the formulations of the invention may vary so long as it is sufficient to provide immediate relief for nasal inflammation, congestion, or allergies. Thus, the final concentration may be about 0.10% to 2.5% v/v citric acid containing solution. In preferred embodiments, the concentration may be about 0.10%, 0.20%, 0.30% 0.40%, 0.5%, 0.60%, 0.70%, 0.80%, 0.80%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.0%-4.0%, 4.0%-5.0%, 5.0%-6.0%, 6.0%-7.0%, 7.0%-8.0%, 8.0%-9.0%, or 9.0%-10.0%, v/v. In more preferred embodiments, the citric acid concentration is about 2%.

Aloe Vera extracts are used in the formulations of the invention for its soothing properties. Thus, the compositions comprise Aloe Vera extracts at concentrations of about 0.5× to 10× Aloe Vera. In preferred embodiments, the concentration may be about 0.50×, 0.60×, 0.70×, 0.80×, 0.90×, or 1.0×. In other preferred embodiments, the concentration may be between about 1.0× to 2.0×, 2.0× to 3.0×, 3.0× to 4.0×, 4.0× to 5.0×, 5.0× to 6.0×, 6.0× to 7.0×, 7.0× to 8.0×, 8.0× to 9.0×, or 9.0× to 10.0× v/v, or increments therebetween. In more preferred embodiments, the Aloe Vera concentration is 1× or 5×.

The formulations of the invention may be pharmaceutical or nutraceutical compositions. These terms may be used interchangeably herein. The compositions may conveniently be administered in unit dosage form and may be prepared by any of the methods well-known in the pharmaceutical art, for example, as described in Remington's Pharmaceutical Sciences, 17^{th} ed., Mack Publishing Co., Easton, Pa. (1985), incorporated herein by reference in its entirety. Pharmaceutical compositions of this invention comprise any of the formulations of the present invention with any pharmaceutically acceptable carrier, adjuvant or vehicle. Pharmaceutically acceptable carriers, adjuvants and vehicles that may be used in the pharmaceutical compositions of this invention include, but are not limited to, ion exchangers, alumina, aluminum stearate, lecithin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Formulations for nasal administration may be solutions in evaporating solvents such as hydrofluorocarbons, and may contain excipients for stabilization, for example, saccharides, surfactants, submicron anhydrous alpha-lactose or dextran, or may be aqueous or oily solutions for use in the form of nasal drops or metered spray. For buccal administration, typical excipients include sugars, calcium stearate, magnesium stearate, pregelatinated starch, and the like. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

Each administration delivers a dose between 50 and 200 μl. Typically, the pharmaceutical compositions of this invention will be administered from about 1 to about 5 times per day. Such administration can be used as a chronic or acute therapy. Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level, treatment may cease. Patients may, however, require intermittent treatment on a long-term basis upon any recurrence of disease symptoms. As the skilled artisan will appreciate, lower or higher doses than those recited above may be required.

In order that the invention described herein may be more fully understood, the following examples are set forth. It should be understood that these examples are for illustrative purposes only and are not to be construed as limiting this invention in any manner.

### EXAMPLES

### Example 1

### Preparation of Exemplary Nasal Formulations

Described here is the preparation of exemplary nasal formulations for treating rhinitis. The formulations have both oil and aqueous phases with emulsifiers. The oil phase was prepared by mixing 450 μL of polysorbate 80 (J.T. Baker, St. Louis, Mo.), 450 μL of 200 proof ethanol (KOPTEC, King of Prussia, Pa.), 36 μL of lemon oil (Aura Carcia, Urbana, Iowa), and 50 μL of clove oil (Puritan's Pride, Oakdale, N.Y.). A total of 13.71 mL of HPLC grade water (EMD, Billerica, Mass.) was incrementally added to the oil phase. The resulting mixture was sonicated between incremental water additions to obtain a homogenous and transparent emulsion. 195 μl of 10% citric solution was added to the resulting mixture to obtain a final concentration of 0.13% in Formulation D:

TABLE I

| Ingredient | Quantity | |
|---|---|---|
| HPLC water | 13.71 | mL |
| Citric Acid (10%) | 195 | μL |
| Polysorbate 80 | 450 | μL |
| Ethanol, 200 proof | 450 | μL |
| Lemon Oil | 36 | μL |
| Clove Oil | 50 | μL |

Another exemplary formulation was prepared using the general method and ingredients presented in Table 1 but the HPLC water contained diluted Aloe Vera. 200× Aloe Vera powder (Lotioncrafter, Eastsound, Wash.) was thus diluted into the formulation at a 1:200 ratio for a final 1× concentration [Formulation B]. Another exemplary formulation was prepared using the general method and ingredients presented in Table I but the 10% citric acid was replaced with freshly squeezed and filtered lemon juice [Formulation C]. Another exemplary formulation was prepared using the general method and ingredients presented in Table I but both the 10% citric acid was replaced with freshly squeezed and filtered lemon juice and it contained 1× Aloe Vera [Formulation A].

### Example 2

### Nasal Formulation Comparisons

The formulations described in Example 1 were shown to provide significant relief from rhinitis. They were compared in live subjects and rated for the speed of onset of action, the duration of nasal relief, and whether or not the subjects experienced rebound congestion.

One male adult with severe rhinitis tested the four formulations by delivering one drop of formulation in each nostril. A washout period of at least one day was applied between testing different formulations.

Formulation A provided the fastest onset of action. Relief was experienced in less than 20 minutes. It lasted for 4 to 6 hours before the symptoms reappeared. The subject continued the treatment with Formulation A for a week without experiencing rebound congestion. The other three formulations started to work between 20-30 minutes and lasted between 2 to 4 hours (Formulation B and C) and 4 to 6 hours (Formulation D). All of the formulations caused some degree of irritation.

In comparison, existing commercial products were compared under the same criteria and found to be inferior. These included lemon-based nasal sprays Gencydo (Weleda AG, Arlesheim, Switzerland), O-Spray (Bioponic Phytoceuticals, Puunene, Hi.), and Ureil Lemon Quince Spray (Uriel Pharmacy, East Troy, Wis.) as well as the capsaicin-based Sinus Buster (Buster Brands, Amityville, N.Y.). None of these commercial products provide the combination of low irritation pain, fast onset and long duration shown by the formulations disclosed herein. Gencydo was found to have an onset time of 20-30 minutes but not as long acting. O-Spray provided instant relief but was very short-acting (a few hours). Ureil was somewhere in between these. Sinus Buster caused a strong irritation.

### Example 3

### Nasal Formulation Optimization

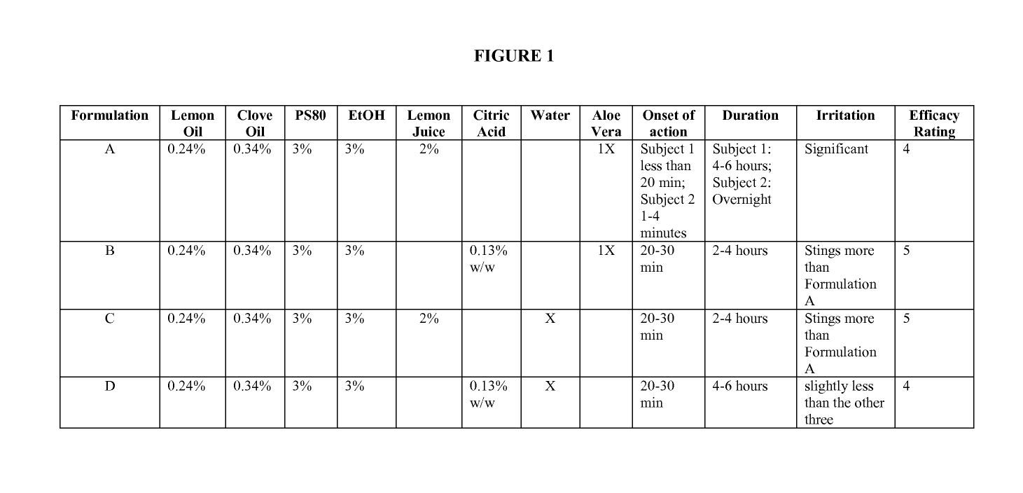

Several formulations of the invention were examined for the time of shown in onset, duration of relief, and relative comfort. The results are provided in FIG. 1. As can be seen, there was variability among the formulations. Longer-term relief was shown with Formulation A (1 week) and at least three days (Formulation B and C). These formulations, however, showed unacceptable amounts of irritation.

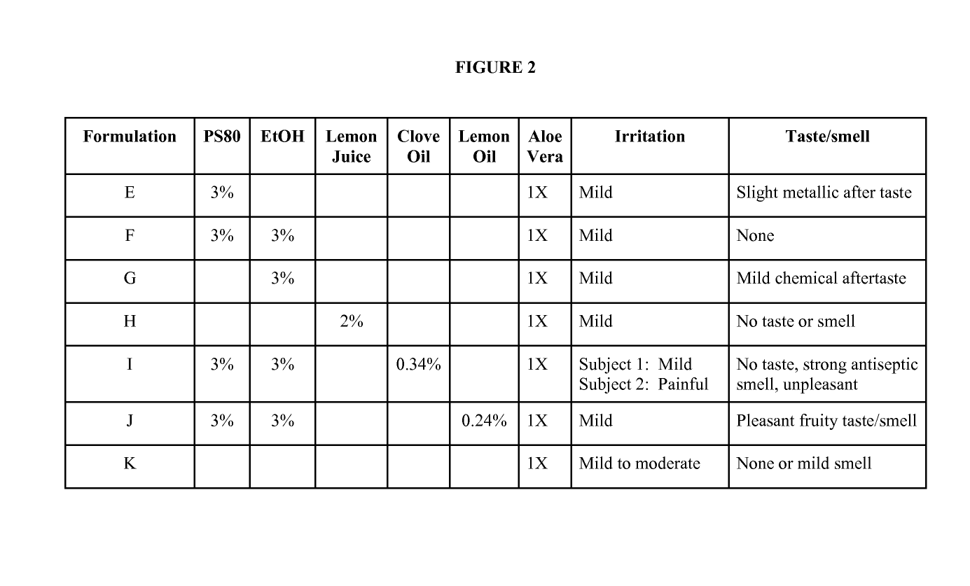

Thus, further formulations were generated and evaluated for the overall experience (irritation, taste, and smell). The results provided in FIG. 2 show that the irritation was caused by the clove oil in the initial formulations.

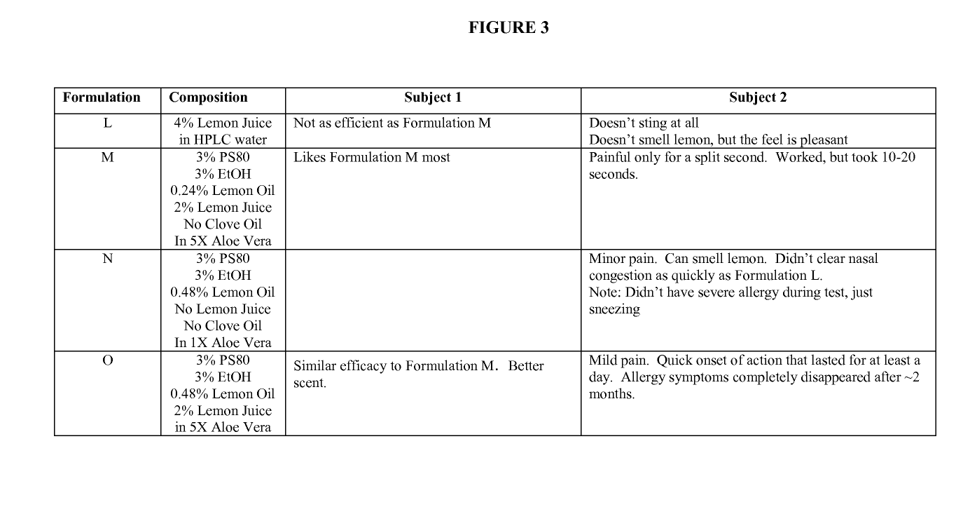

Based on these results, the formulations were further optimized and the results are presented in FIG. 3. These further optimizations eliminated clove oil and increased the Aloe Vera concentration to 5×. Another optimization increased the Lemon oil to 0.48% (Formulation O). This formulation was tested on 6 additional subjects. Each of them reported that the formulation was highly effective against allergy symptoms. Two subjects reported that the formulation worked within a few minutes, and that two doses per day sufficiently treated their allergy symptoms. One subject reported that the formulation worked in a couple of minutes, and that the effects lasted approximately 6 hours. In addition, she stated that she liked the smell and the fact that this formulation is all-natural. Another subject reported that the effects last about 2-3 hours and the formulation stops running noses in 5-15 min. In addition, she likes the citrus fragrance and cool sensation. Surprisingly, one subject was completely free from allergy symptoms after approximately 2 months of usage.

### Example 4

### Addition of Kushen Extract

Formulation P was prepared with 3% v/v polysorbate 80, 3% ethanol, 0.48% Lemon Oil, 2% Lemon Juice, 5× Aloe Vera, and 0.3% Kushen. The formulation was filtrated through 0.2 um filter. The formulation has rapid onset of action, low irritation and had long duration of action.

All publications and patent documents disclosed or referred to herein are incorporated by reference in their entirety. The foregoing description has been presented only for purposes of illustration and description. This description is not intended to limit the invention to the precise form disclosed. It is intended that the scope of the invention be defined by the claims appended hereto.

What is claimed:

1. A composition for nasal administration, comprising
a. about 0.10% to 10% v/v lemon oil;
b. about 1.0% to 10% v/v emulsifier;
c. about 1.0% to 10% v/v alcohol; and
d. about 0.10 to 2.5% v/v of a citric acid-containing solution;
wherein said composition reduces the severity of rhinitis.

2. The composition of claim 1, further comprising between about 0.5× and 10× Aloe Vera.

3. The composition of claim 1, wherein said citric acid containing solution contains flavonoids.

4. The composition of claim 3, wherein said citric acid containing solution is lemon juice at a concentration of about 2.0% v/v.

5. The composition of claim 1, wherein said lemon oil is at a concentration of about 0.24% v/v.

6. The composition of claim 1, wherein said lemon oil is at a concentration of about 0.48% v/v.

7. The composition of claim 2, wherein said lemon oil is at a concentration of about 0.24% v/v.

8. The composition of claim 2, wherein said lemon oil is at a concentration of about 0.48% v/v.

9. The composition of claim 3, wherein said lemon oil is at a concentration of about 0.24% v/v.

10. The composition of claim 3, wherein said lemon oil is at a concentration of about 0.48% v/v.

11. The composition of claim 4, wherein said lemon oil is at a concentration of about 0.24% v/v.

12. The composition of claim 4, wherein said lemon oil is at a concentration of about 0.48% v/v.

13. The composition of claim 11, wherein said Aloe Vera is at about a 1× concentration.

14. The composition of claim 12, wherein said Aloe Vera is at about a 1× concentration.

15. The composition of claim 11, wherein said Aloe Vera is at about a 5× concentration.

16. The composition of claim 12, wherein said Aloe Vera is at about a 5× concentration.

17. The composition of claim 1, wherein said emulsifier is selected from the group consisting of egg yolk (lecitihin), mustard (mucilage), soy lecithin, pickering stabilization, sodium stearoyl lactylate, DATEM (Diacetyl Tartaric Acid Ester of Monoglyceride), anise, emulsifying wax, cetearyl alcohol, polysorbate 20, and ceteareth 20.

18. The composition of claim 1, wherein said alcohol is ethanol, isopropanol, or benzyl alcohol.

19. The composition of claim 1, wherein
a. said lemon oil concentration is about 0.48%;
b. said emulsifier is polysorbate 80 at a concentration of about 3.0%;
c. said alcohol is ethanol at a concentration of about 3.0%;
d. said citric acid-containing solution is lemon juice at a concentration of about 2.0% v/v; and
e. further comprising Aloe Vera at a concentration of about 5×.

20. A method for treating rhinitis in a subject, comprising administering the composition of claim 1 to said subject.

21. A method for treating rhinitis in a subject, comprising administering the composition of claim 2 to said subject.

22. A method for treating rhinitis in a subject, comprising administering the composition of claim 19 to said subject.
